(19) United States
(12) Patent Application Publication
(10) Pub. No.: US 2005/0244811 A1
(43) Pub. Date: Nov. 3, 2005

(54) Matrix array nanobiosensor

(75) Inventors: Prabhu Soundarrajan, Austin, TX (US); Valerie Ginsberg, Austin, TX (US); Zvi Yaniv, Austin, TX (US)

Correspondence Address: Kelly K. Kordzik;Winstead Sechrest & Minick P.C., P.O. Box 50784, Dallas, TX, 75201, US

(73) Assignee: Nano-Proprietary, Inc., Austin, TX (US)

(21) Appl. No.: 11/011,645

(22) Filed: Dec. 14, 2004

Related U.S. Application Data
(63) Continuation-in-part of application No. 10/952,669, filed on Sep. 29, 2004.
(60) Provisional application No. 60/529,683, filed on Dec. 15, 2003.
(60) Provisional application No. 60/531,819, filed on Dec. 22, 2003.

(51) Int. Cl. C12Q 1/00; C12Q 1/68; C12M 1/34

(52) U.S. Cl. 435/4; 435/287.1

(57) ABSTRACT

An apparatus for detecting multiple analytes comprising an array of nanobiosensors, each comprising a biological entity immobilized onto carbon nanotubes, wherein a plurality of the nanobiosensors in the array have unique biological entities, wherein a first one of the plurality of nanobiosensors has a first biological entity immobilized onto carbon nanotubes, and wherein a second one of the plurality of nanobiosensors has a second biological entity immobilized onto carbon nanotubes, the first biological entity is unique relative to the second biological entity.

### CROSS-REFERENCE TO RELATED APPLICATIONS

The present application claims priority to U.S. Provisional Patent Application Ser. Nos. 60/529,683 and 60/531,819, which are hereby incorporated by reference herein. The present application is a continuation-in-part application of U.S. patent application Ser. No. 10/952,669, which is hereby incorporated by reference herein.

### TECHNICAL FIELD

The present invention relates in general to biological sensors, and in particular, to biological sensors arranged in a matrix array.

### BACKGROUND INFORMATION

The simultaneous monitoring for multiple analytes (liquids and gases) has diversified applications in various domains like metabolic monitoring, chemical, biological warfare detection, gas sensing, etc. Most present day sensors have considerable limitations in monitoring more than one analyte due to the problems of cross-sensitivity and interference from other compounds. This limitation is a disadvantage with continuous detection of multiple analytes. Some of the multi-analyte systems do not have sufficient miniaturization for in vivo or other sensitive applications. There is a lack of unified sensor arrays that can monitor both gases and liquids simultaneously.

There have been several reports on the development of biosensor arrays using different methodologies. A common biosensor format for an enzyme based biosensor array to monitor fruit quality was reported (Biosensors & Bioelectronics (2003), 18(12), 1429-1437). Pectin was used as the immobilization matrix for the sensors, but the methodology of immobilization was “drop and dry mechanism” which did not yield good sensitivity.

A two enzyme biosensor array for characterization of wastewaters incorporating tyrosinase and horseradish peroxidase (HRP) or cholinesterase-modified electrodes were combined on the same array (Analytical and Bioanalytical Chemistry, Vol. 376, Issue 7, 2003, p. 1098). The performances of bi-enzyme biosensor arrays in the batch mode and in the flow-injection system were discussed.

A multifunctional bio-sensing chip was reported based on the electrochemiluminescent (ECL) detection of enzymatically produced hydrogen peroxide (Marquette, Christophe A.; Degiuli, Agnes; Blum, Loic J., Biosensors & Bioelectronics (2003), 19(5), 433-439). Six different oxidases specific for choline, glucose, glutamate, lactate, lysine and urate were non-covalently immobilized on in the array sensor but the limit of detection was only towards hydrogen peroxide.

Pin printed biosensor arrays (PPBSA) were reported by pin printing protein-doped xerogels (Cho, Eun Jeong; Tao, Zunyu; Tehan, Elizabeth C.; Bright, Frank V., Analytical Chemistry (2002), 74(24), 6177-6184). The sensor was able to detect glucose and oxygen simultaneously. The overall array-to-array response reproductibilities are around 12%, which limits the long time stability of the sensor.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates an embodiment of the present invention;

FIG. 2 illustrates an embodiment of the present invention;

FIG. 3 illustrates electronic circuitry for input and output of information from embodiments of the present invention;

FIG. 4 illustrates responses from nine sensor elements;

FIG. 5 illustrates responses of embodiments of the present invention;

FIG. 6 illustrates a response of a matrix biosensor towards ascorbic acid;

FIG. 7 illustrates a graph of responses of an exemplary embodiment of the present invention;

FIGS. 8-9 illustrate graphs of operation of a sensor configured in accordance with the present invention;

FIG. 10 illustrates an alternative embodiment of the present invention;

FIGS. 11A and 11B illustrate further details of an alternative embodiment of the present invention;

FIG. 12 illustrates a matrix array embodiment of the present invention;

FIG. 13 illustrates active circuits for addressing embodiments of the present invention in an array;

FIG. 14 illustrates a matrix array configuration in accordance with an embodiment of the present invention; and

FIG. 15 illustrates an alternative embodiment of the present invention.

### DETAILED DESCRIPTION

In the following description, numerous specific details are set forth such as specific memory array configurations, etc. to provide a thorough understanding of the present invention. However, it will be obvious to those skilled in the art that the present invention may be practiced without such specific details. In other instances, well-known circuits have been shown in block diagram form in order not to obscure the present invention in unnecessary detail. For the most part, details concerning timing considerations and the like have been omitted inasmuch as such details are not necessary to obtain a complete understanding of the present invention and are within the skills of persons of ordinary skill in the relevant art.

Refer now to the drawings wherein depicted elements are not necessarily shown to scale and wherein like or similar elements are designated by the same reference numeral through the several views.

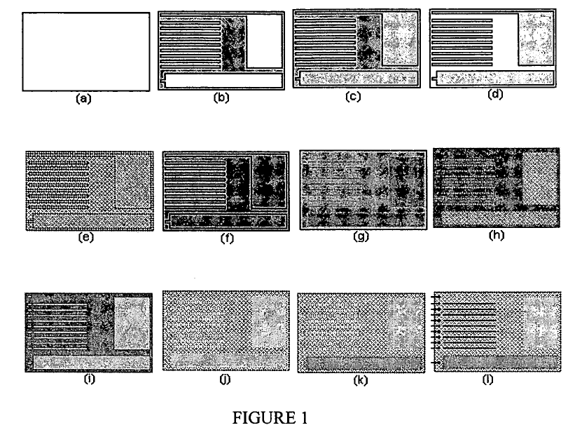

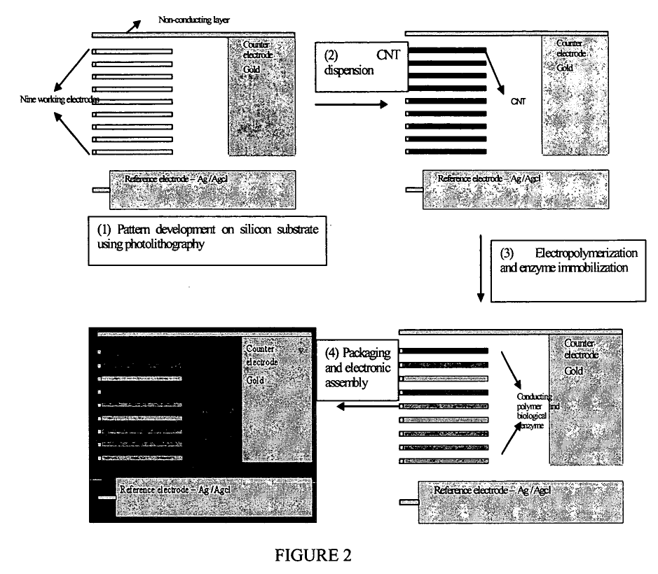

One embodiment of the present invention illustrated in FIGS. 1-2 is a matrix array nanobiosensor for monitoring nine analytes, comprising carbon nanotubes, conducting polymers, biological enzymes, nanoparticles and other nanoscale materials as sensor elements (working electrodes) built in a three electrode electrochemical system. The sensor element is miniaturized sufficiently to operate with very small amounts of analyte and applicable to sensitive applications. An inexpensive photolithographic fabrication process is employed for substrate preparation, and the sensor has miniaturized electronics that can couple between the individual nine electrodes for efficient detection.

The sensor may find applications in domains like metabolic monitoring (glucose, lactose, fructose, urea, uric acid, phenol, alcohols, ascorbic acid, hydrogen peroxide, phospholipids and other metabolites), chemical warfare detection (sarin, tabun, soman, hydrogen cyanide, cyanogens chloride, mustard, chlorine and other chemical warfare agents), biological warfare agents (ricin, polypeptides and others), potential chemical biological warfare agents (PCB's such as organophosphates, DMMP, malathion, ethion, parathion, paraozon and others), DNA hybridization, gas monitoring (toxic gases like CO, SO₂, NO, NO₂, NH₃, H₂S and others), and metals (mercury, arsenic and others). The matrix nanobiosensor can be used to detect both gases and liquid multiple analytes.

Photolithographic Fabrication of a Matrix Nanobiosensor:

Referring to FIG. 1, using chemical vapor deposition (CVD), 1 μm of silica dioxide is deposited onto a silica wafer (step a). A shadow mask (step b) having a desired pattern is placed onto the substrate, and 100 Å of chromium, then 500 Å of gold are deposited via electron beam deposition (step c). The shadow mask is removed (step d). By the process of CVD, 0.3 μm of silica nitride is deposited (step e). The substrate is coated with photo-resist (step f). Using a mask (step g), a photolithographic process is used to pattern the substrate (step h). The exposed silica nitride (SiI₃N₄) pattern is removed via reactive ion etching (RIE) (step i). The photo-resist is removed (step j), and Ag/AgCl paste (obtained from Gwent Electronic Materials Ltd, U.K) is screen-printed onto the reference electrode pattern (step k). The Ag/AgCl can also be electrodeposited as a layer of silver at −200 mV followed by a chloride layer at +200 mV through potentiostatic methods. Electrical contacts are then soldered onto each electrode (step l).

Sensor Fabrication:

The matrix nanobiosensor substrate developed by photolithography fabrication as described in FIG. 1(l) is as shown in step (l) of FIG. 2, with nine individual working electrodes 201, a reference electrode 203 (screen printed Ag/AgCl paste) and a counter electrode 202 (gold).

In step 2 (FIG. 2), the carbon nanotubes (CNT) 204 are dispensed (sprayed or screen printed) onto the nine working electrodes 201 using a suitable mask (not shown).

Carbon nanotube paste electrodes (0.5 cm²) were prepared by mixing 50% by weight of carbon nanotubes with 43% by weight of organic (or inorganic) vehicle and 7% by weight of glass frit in a mortar and pestle for 30 minutes followed by grinding in a three roll mill for 20 minutes to disperse the clusters in the mixture. The inorganic vehicle was purchased from Cotronics Corp., Brooklyn, N.Y., USA. The substrate was then baked at 100° C. for 10 minutes in an oven and cooled at room temperature. Different weight percentages of carbon nanotubes can also be employed for the electrode preparation. The prepared carbon nanotube paste electrodes can be fired (hard baked) to remove the organic vehicle and activated using a tape.

Carbon nanotube spray electrodes (0.5 cm²) were prepared by dissolving a known quantity of carbon nanotubes (e.g., 0.1 g) in 20 ml isopropyl alcohol, followed by ultrasonication for 5 minutes, and spraying the solution onto the substrate (silicon substrate, vacuum evaporated with 20 Å chromium and 500 Å gold). The spray electrode was then baked at 100° C. for 10 minutes in an oven and cooled at room temperature.

The carbon nanotubes that can be used with this invention can also be prepared by a chemical vapor deposition process including a catalyst (e.g., nickel, copper, cobalt, iron) and a carbon source (e.g., acetylene, ethylene, methane and other hydrocarbons), or other methods known to one skilled in the art.

In step 3 (FIG. 2), the electrochemical polymerization and enzyme immobilization was carried onto the carbon nanotube electrodes in situ (applicable to all the electrodes discussed above) by the oxidation of aniline (0.1 M), 1 mg/ml of different biological enzymes 205 in a solution containing 0.2 M H₂SO₄ in a pH 7.0 buffer solution (note that the enzyme solution preparation varies for different enzymes based on the activity of the enzyme at a particular pH). A potential window of −1 V to 1 V was employed for the electropolymerization and immobilization with a scan rate of 50 mV/s for 10 cycles. The different enzymes 205 employed for the nine sensor elements 201 can be selected with regards to the specific analyte in Table 1, though the enzyme systems that can be used in the matrix nanobiosensors are not limited to the list in Table 1.

TABLE 1

| Analyte | Enzyme |
|---|---|
| Glucose | Glucose oxidase, Glucose dehydrogenase |
| L-Lactate | Lactate oxidase, Lactate dehydrogenase |
| Phenol, catechol, | Tryosinase (polyphenol oxidase) |
| p-cresol, m-cresol, | |
| Atrazine | |
| Urea | Urease |
| Ascorbic acid | Ascorbic oxidase |
| Choloestrol | Choloestrol oxidase, Choloestrol dehydrogenase |
| Fructose | Fructose dehydrogenase |
| Lipids, Triglycerides | Lipase |
| Uric acid | Uricase |
| Choline, Lecithin | Choline oxidase |
| Hemoglobin | Pepsin |
| Glutamate | Glutamate oxidase |
| Alcohol | Alcohol dehydrogenase, Alcohol oxidase |
| Carbon monoxide | Carbon monoxide dehydrogenase |
| Sucrose | Invertase, mutarotse |
| Malate | Malate oxidase, NADH oxidase |
| Lycine | Lycine oxidase |
| Glycerol | Glycerol dehydrogenase |
| Citrate and pyruvic | Citrate lyase and pyruvate oxidase |
| acid | |
| Sulphite | Sulphite oxidase |
| Gelatinized starch | Amyloglucosidase, α-amylase, glucose oxidase |
| Penicillin | Immobilized penicillin |
| Tannin | Laccase |
| Formate | Formate dehydrogenase |
| Hydrogen peroxide | Horseradish peroxidase |

Electropolymerization and enzyme immobilization of polypyrrole and these enzymes was carried out by the oxidation of pyrrole (0.1 M) in a solution containing 0.1 M NaClO₄ in a pH 7.0 buffer solution under the same electrochemical conditions. The electrodes were then washed with water and dried in air. Other conducting polymers can also be employed in these matrix nanobiosensors. Additionally, other biological entities such as antibodies, nucleic acids, aptamers, etc., can be immobilized onto the nanotubes using similar methods.

In step 4 (FIG. 2), the sensor element is placed in a suitable electronic housing and coupled to external electronics by electrodes 206. The sensor element is also filled with an electrolye necessary for the specific electrochemical reaction based on the analyte (liquid or gaseous) to be detected. The nine working electrodes 201, counter electrode 202 and reference electrodes 203 are coupled to the drive electronics (discussed below), which is a potentiostatic circuit necessary for any three electrode electrochemical system.

Electronic Drive Assembly:

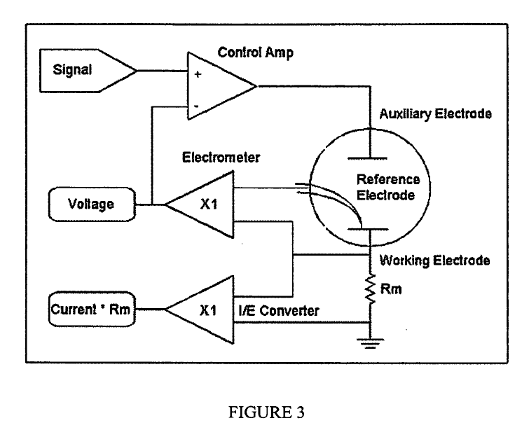

Referring to FIG. 3, the proposed electronics to connect to the above-described array consists of a multi-channel sensor driver. It is capable of operating in either pulsed or continuous scan modes, uses a low-power microprocessor and is programmable. The processor drives signals out to a D/A converter that drives all sensors and a D/A converter that reads sensor outputs. A controlled-impedance op-amp for each sensor, with multiplexer, handles the periodic reading of each of the sensors in turn. Software in the processor computes the present background currents and runs an algorithm to detect peaks above the expected background. The peaks are compared with patterns for known analytes, after being first corrected for chemistry-induced peak shifts. Results are presented in both qualitative and quantitative output channels. In the final system, the quantitative channel will be optional, with presence of an analyte indicated by LED display or other suitable devices. False positives/negatives can be efficiently eliminated due to the peak current at a unique redox potential of the analyte which presents a considerable improvement over simple resistive based sensors arrays. The components of the electronic assembly may be as follows:

(a) The Electrometer

The electrometer circuit 301 measures the voltage difference between the reference 203 and working 201 electrodes. Its output has two major functions: it is the feedback signal in the potentiostat circuit, and it is the signal that is measured whenever the cell voltage is needed. An ideal electrometer has zero input current and infinite input impedance. Current flow through the reference electrode 203 can change its potential. In practice, all modern electrometers have input currents close enough to zero that this effect can usually be ignored. Two important electrometer characteristics are its bandwidth and its input capacitance. The electrometer bandwidth characterizes the AC frequencies the electrometer 301 can measure when it is driven from a low impedance source. The electrometer bandwidth is higher than the bandwidth of the other electronic components in the potentiostat. The electrometer input capacitance and the reference electrode resistance form an RC filter. If this filter's time constant is too large, it can limit the effective bandwidth of the electrometer and cause system instabilities. Smaller input capacitance translates into more stable operation and greater tolerance for high impedance reference electrodes.

(b) The Current to Voltage Converter

The current to voltage (I/E) converter 302 in the simplified schematic measures the cell current. It forces the cell current to flow through a current measurement resistor, Rm. The voltage drop across Rm is a measure of the cell current. A number of different Rm resistors can be switched into the I/E circuit 302 under computer control. This allows measurement of widely varying of currents, with each current measured on using an appropriate resistor. An “I/E autoranging” algorithm is often used to select the appropriate resistor values. The I/E converter's bandwidth depends strongly on its sensitivity. Measurement of small currents requires large Rm values. Stray (unwanted) capacitance in the I/E converter 302 forms an RC filter with Rm, limiting the I/E bandwidth.

(c) The Control Amplifier

The control amplifier 303 is a servo amplifier. It compares the measured cell voltage with the desired voltage and drives current into the cell to force the voltages to be the same. Note that the measured voltage is input into the negative input of the control amplifier 303. A positive perturbation in the measured voltage creates a negative control amplifier output. This negative output counteracts the initial perturbation. This control scheme is known as negative feedback. Under normal conditions, the cell voltage is controlled to be identical to the signal source voltage.

(d) The Signal

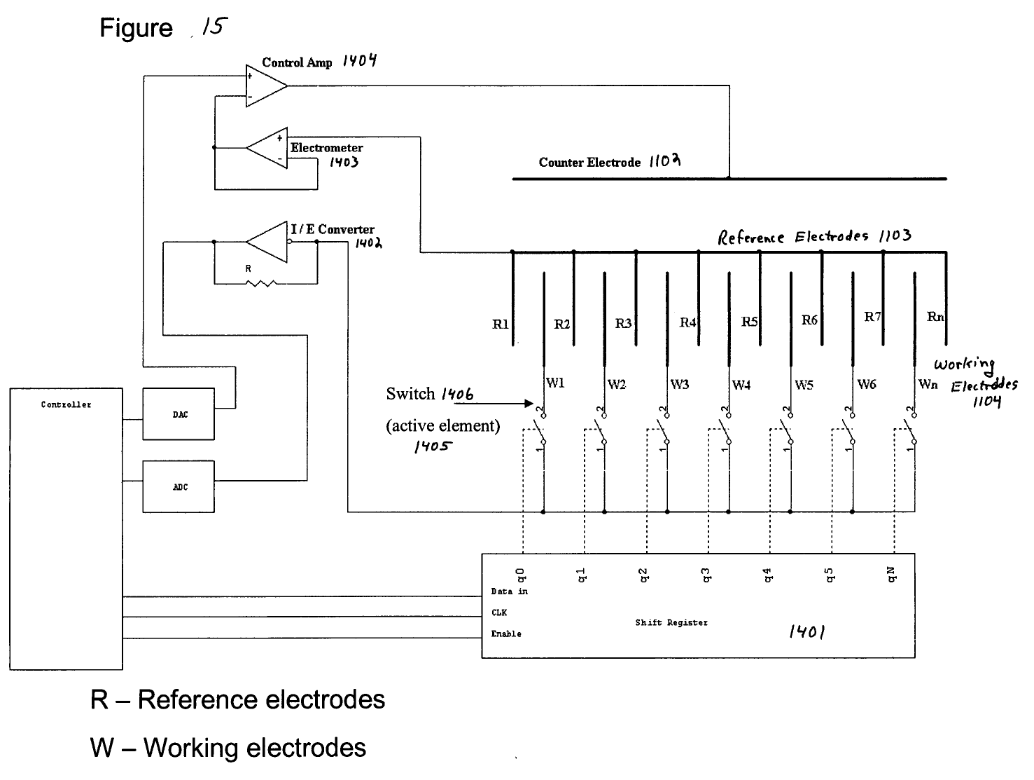

The signal circuit 304 is a computer controlled voltage source. It is generally the output of a digital to analog (D/A) converter (see DAC in FIG. 15) that converts computer generated numbers into voltages. Proper choice of number sequences allows the computer to generate constant voltages, voltage ramps, and even sine waves at the signal circuit output. When a D/A converter is used to generate a waveform such as a sine wave or a ramp, the waveform is a digital approximation of the equivalent analog waveform. It contains small voltage steps. The size of these steps is controlled by the resolution of the D/A converter and the rate it at which it is being updated with new numbers.

Mechanism of Sensing:

One of the mechanisms of sensing as described previously is electrochemical based. The qualitative sensing is achieved by cyclic voltammetry, which is used to characterize the unique amperometric oxidative potential. The quantitative sensing is carried out by chronoamperometric measurements at the fixed characteristic potential determined by cyclic voltammetry. The liquid phase sensors require a small amount of analyte (in micromolar range) and the gas phase sensors are provided with a hydrophobic membrane and a liquid or solid electrolyte. The solid electrolyte can be any anionic exchange membrane (e.g., nafion), nanoporous silica (e.g., xerogels, hydrogels).

The enzymes may be immobilized into the nanotubes using a cyclic voltammetric (CV) technique (here the voltage is varied in steps, typically swept between −1V to +1V and reverse for one loop). Nine different enzymes (E) may be immobilized onto the sensor elements using CV to form the sensor array. The nine different enzymes are selected to have a unique reaction with nine different analytes (A) [example: Glucose oxidase (E) for glucose (A)]. When the analyte comes in contact with the sensor, the matrix is turned on by the electronics (the background electrochemical process in the electronics is CV) and the CV has a unique redox peak for each of the analytes resulting from the enzyme (E) vs analyte (A) reaction. Based on the redox peak for each analyte obtained, the software calibrates the concentration levels of the analyte.

Chronoamperometry operates by fixing a constant voltage and gives out current vs time plots. A characteristic voltage is fixed for each analyte obtained from the previous CV run. An advantage of this technique is that the measurements can be done real time and faster compared to the scan method in the CV.

The response of the matrix nanobiosensor towards hydrogen peroxide is shown in equation (1) below. As an example, ten enzymatic schemes are illustrated. It can be seen than hydrogen peroxide is a by-product of the enzymatic reaction in equations (2) through (6).

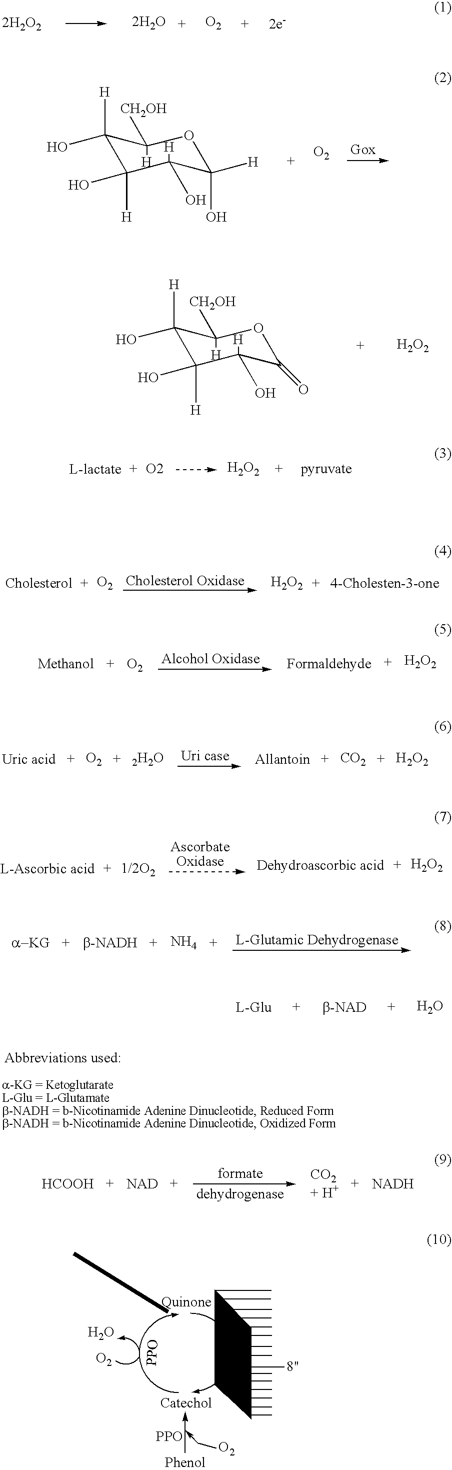

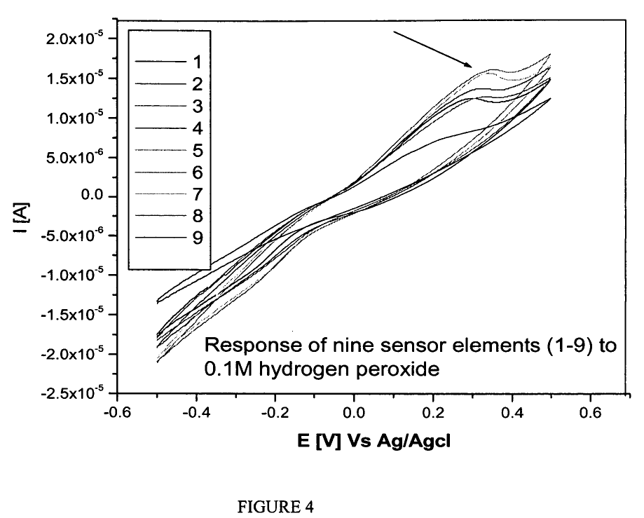

The response of the individual nine elements of the matrix nanobiosensor towards hydrogen peroxide is shown in FIG. 4. While pristine carbon nanotubes can oxidize hydrogen peroxide, the presence of an enzyme and the conducting polymer is a requirement for enzymatic biosensing applications according to the present invention. As can be seen, there is a small variation in the peak responses and amperometric oxidation voltages in the nine individual elements, but the anodic oxidation potential is still much lower than reported in the literature. Previous reports indicate the development of glucose sensors by the addition of palladium, copper, iridium or ruthenium into carbon paste electrodes with glucose oxidase. (S. A. Miscoria, G. D. Barrera, G. A. Rivas; Electroanalysis, 14, 300, 2003), and phenol sensors with the incorporation of iridium microparticles into carbon paste matrices with polyphenol oxidase (M. D. Rubianes, G. A. Rivas, Electroanalysis, 12, 1159). These approaches do not incorporate a conducting polymer matrix and involve the mechanical mixing of the enzyme into the carbon paste matrix.

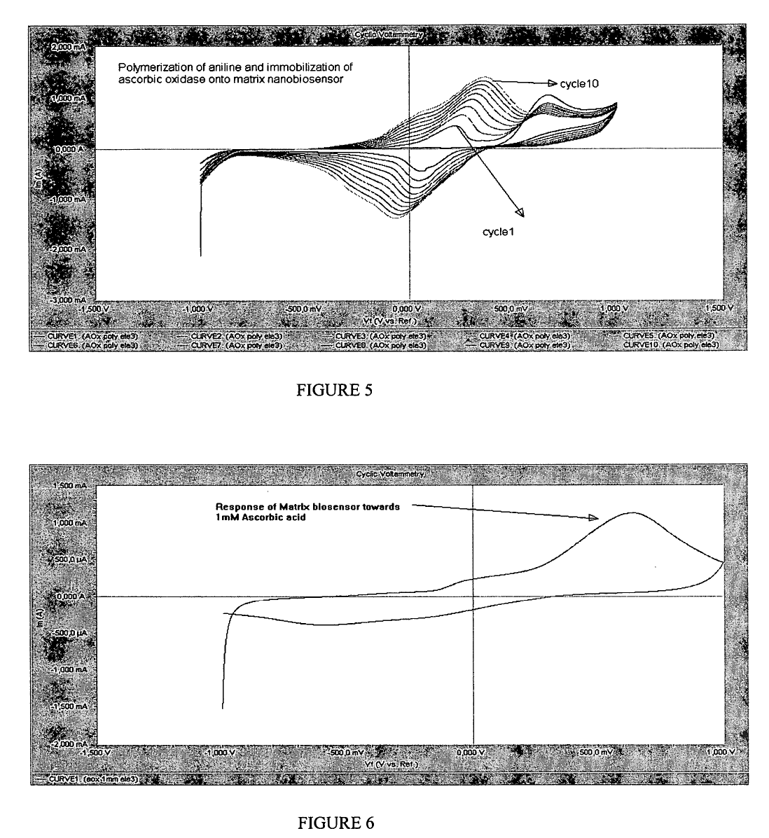

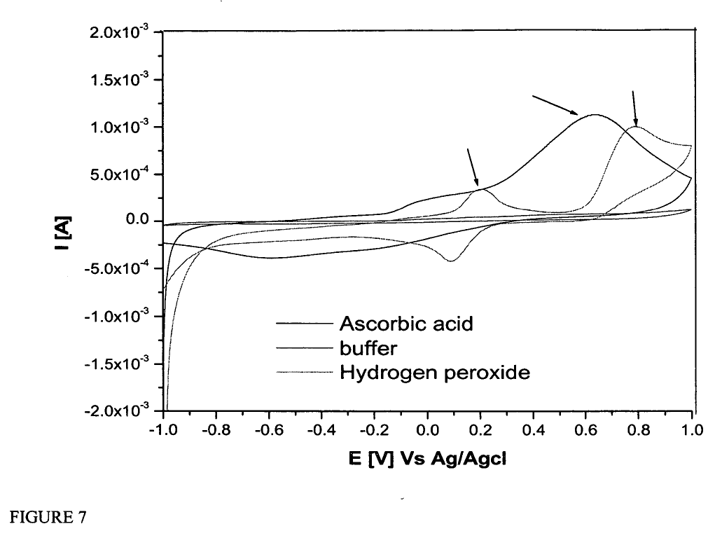

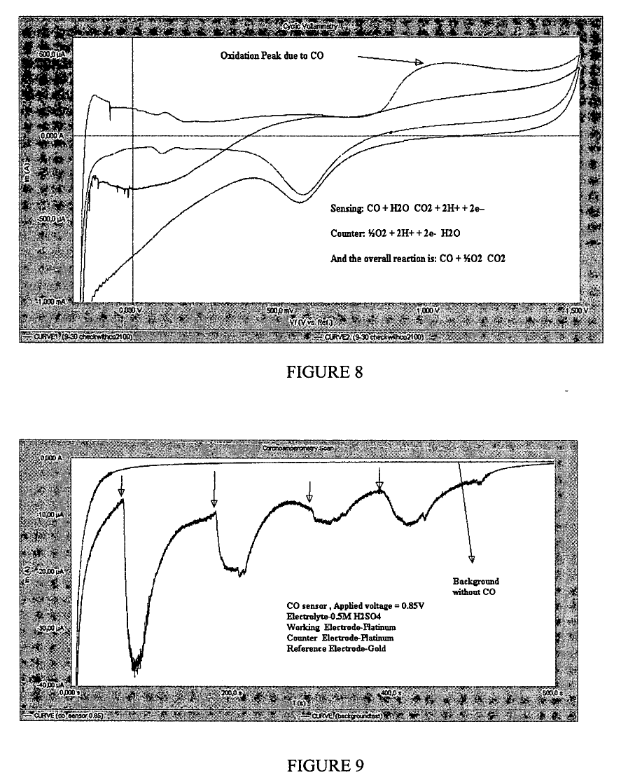

Enzymatic schemes illustrated in equations (7) to (10) do not release hydrogen peroxide as a result of biochemical reaction, but the analytes can be detected by monitoring other products namely dehydroascorbic acid (ascorbic oxidase −7), glutamate (L-glutamic dehydrogenase −8), CO₂ (formate dehydrogenase −9), quinone (polyphenol oxidase −10). This invention is not limited to the ten enzymes illustrated in the reaction schemes or in Table 1, but any redox active enzyme systems can be implemented. As an example, the cyclic voltammogram of the polymerization of aniline (0.1 M in 0.2 M H₂SO₄) in situ with ascorbic acid (equation (7)) onto the carbon nanotube electrode is shown in FIG. 5. The response of the peak response current (0.6 V) due to the oxidation of ascorbic acid into dehydroascorbic acid is shown in FIG. 6. The selectivity of the sensor is illustrated in FIG. 7, where the oxidative peaks are clearly distinguishable for hydrogen peroxide. This eliminates interference from ascorbic acid and also provides higher selectivity.

Similar results were obtained for other enzymatic systems. The matrix nanobiosensor did not have any interference from consecutive sensor elements, though different enzymes are immobilized onto nine individual elements.

Stability of the Matrix Nanobiosensor:

The matrix nanobiosensor array was stable over a number of assays (over hundred assays), the lifetime of the sensor is a function of the activities of the enzyme. The conducting polymer matrix in the nanobiosensor provides a good stability for the enzymes in the nanotube matrix. The specific enzyme stability based on its biochemical activity is given in Table 2. Further, the enzyme stabilization can extend the lifetime of the sensors.

TABLE 2

| | Length of | |
|---|---|---|
| | Stability | Storage Temperature (° C.) and Application |
| Biological enzyme | (Days) | Domains |
| Acetylcholinesterase | 76 | 37 |
| | | Pesticide detection grain, fruit and water |
| Alcohol Oxidase | 162 | 37 |
| | | Alcohol detection brewing, fermentation, breath |
| | | analysis |
| Catalase | 22 | 37 |
| Cholesterol Oxidase | 16 | 37 |
| | | Cholesterol level testing |
| Choline Oxidase | 15 | 37 |
| | | Choline esterase activity and phospholipids |
| | | determination |
| Diaphorase | 150 | 37 |
| | | Hygene test (NADH, ATP) |
| Fructose Dehydrogenase | 155 | 4 |
| | | Fruit and wine analysis |
| Fructose Dehydrogenase | 155 | 25 |
| Galactose Oxidase | 17 | 37 |
| | | sugar analysis in food |
| β-Galactosidase | 65 | 25 |
| | | Disease diagnosis for food allergies |
| Glucose Dehydrogenase | 13 | 37 |
| | | Glucose sensors healthcare and agrifood sector |
| Glucose-6-Phosphate | 150 | 37 |
| Dehydrogenase | | Hygene test |
| Glutamate Dehydrogenase | 665 | 22 |
| | | Ammonia in water, healthcare glutamate in food |
| | | suffs and healthcare neurotransmitter analysis |
| Glycerol-3-Phosphate Oxidase | 37 | 15 |
| | | triglyceride analysis healthcare |
| Hexokinase | 150 | 37 |
| | | hygene test |
| Horseradish Peroxidase | 50 | 37 |
| | | glucose sensors healthcare and agrifood sectors |
| β-Hydroxybutyrate | 36 | 37 |
| Dehydrogenase | | detection of ketone bodies human healthcare |
| Lactate Dehydrogenase | 190 | 25 |
| | | animal healthcare and sport performance marker |
| Lactate Oxidase | 300 | 37 |
| | | animal healthcare and sport performance marker |
| Malate Dehydrogenase | 20 | 37 |
| | | Wine quality measurement and human healthcare |
| NADH | 182 | 22 |
| | | Any dehydrogenase based sensor or assay |
| Pyruvate Kinase | 150 | 37 |
| | | hygene testing and sports performance marker |
| Serine Protease | 56 | 66 |
| | | laundry products |
| Uricase | 10 | 37 |
| | | uric acid determination human healthcare |

Matrix Nanobiosensor for the Detection of Toxic Gases:
Carbon Monoxide (CO) Sensor:

The working electrode is composed of a nanostructured platinum material namely platinum nanoparticles or carbon nanotubes electroplated with platinum nanoparticles. The main reason for the employment of platinum as the working electrode is its known catalytic oxidation of carbon monoxide. The coating of the platinum nanoparticles onto the carbon nanotubes increases the surface catalytic activity of the working electrode towards CO yielding a higher sensitivity. The counter electrode is composed of a metal (e.g., platinum, gold, etc.) and the reference electrode (e.g., Ag/AgCl). The electrolyte constitutes a strong acidic electrolyte (e.g., M H₂SO₄), and the sensor is enclosed in a hydrophilic semi-permeable membrane.

The electrochemical reaction involving the detection of carbon monoxide using the sensor is,

Reaction at the sensing electrode: CO+H₂O →CO₂+2H++2e−
Reaction at the counter electrode: 1/2O₂+2H++2e−→H₂O
And the overall reaction: CO+ 1/2O₂→CO₂

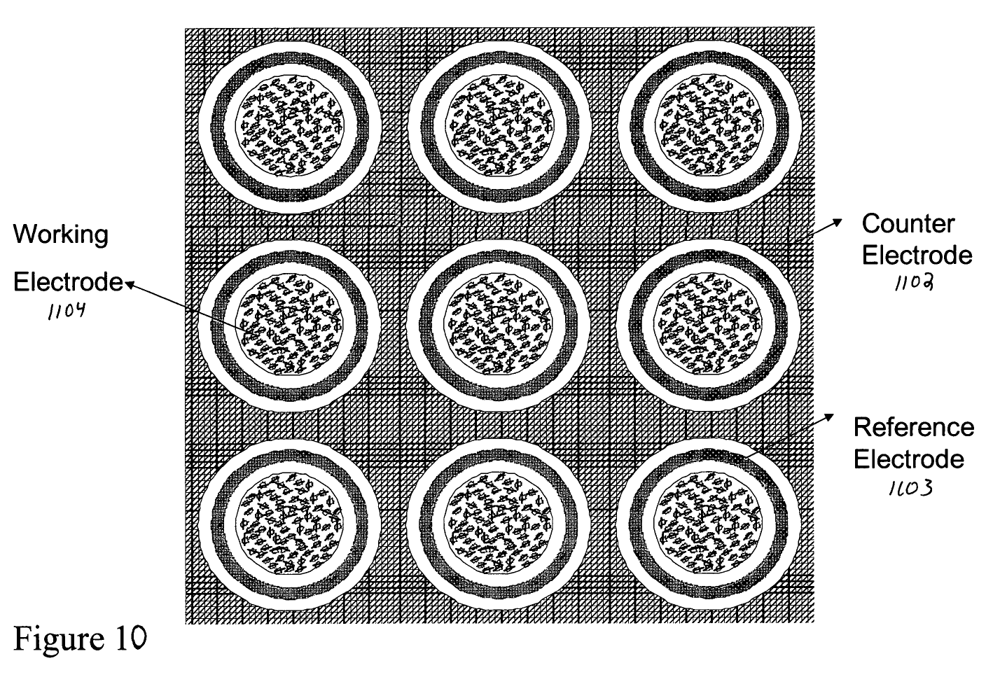

The cyclic voltammogram of the oxidation of CO at platinum electrodes using the sensor is shown in FIG. 8, wherein the lower curve does not show a characteristic peak due to the absence of CO. When CO is exposed to the sensor, there is a characteristic oxidation peak at around 0.85 V. The proposed approach of the carbon nanotube-platinum nanoparticle composites will have a higher sensitivity and a lower oxidation potential as observed for the hydrogen peroxide sensors due to the involvement of carbon nanotubes. FIG. 9 describes the chronoamperometric response of the sensor to the exposure of CO. The sensor has a quick response time and provides reliable measurements since the oxidation peak of 0.85 V is characteristic towards the oxidation of CO by platinum.

The embodiments discussed above describe the fabrication of a nine element matrix for the development of nanobiosensors. The previous design electronics incorporated the individual driving of each sensor element along with the reference and counter electrodes. Though the design is a good development over the single element biosensor, there may be some difficulty extending this design for the development of n×n sensor elements. The previous design also incorporated a planar structure, wherein the reference, counter and working electrodes are in a silicon chip oriented on the X-Y plane. The nanobiosensor can be extended to detect hundreds of analytes if the biosensor substrate is linear. There is a requirement of an optimized design and electronic driving to extend the diversity of the nanobiosensors.

The following embodiments provide an alternative design to the nanobiosensors. Disclosed are the following:

1) A linear design approach for the three electrode system (working, counter, reference electrodes).

2) The specific placement of the reference electrode in close proximity of the working electrode in a single element and a matrix array form.

3) The design of a semi-permeable, hydrophobic membrane on the area of the counter electrode, and design of the counter electrode in close proximity to the reference and working electrode in the electrolyte separate from the membrane. This is of high significance in the development of electrochemical gas sensors.

4) Development of miniaturized electronics which enable the driving of the linear and matrix array elements. The sensor electronics enable cyclic voltammetric and chroamperometric measurements in the sensor without the standard laboratory based potentiostat.

5) Design of an active matrix (analogous to the active matrix in liquid crystal displays) for the nanobiosensor application, which can enable the development of n×n sensor elements in a compact area. This invention is the first reported for the development of active matrix systems for the electrochemical (three electrode) systems. The electronics developed for the active matrix employs a shift register and can drive all the sensor elements in the active matrix.

Electrochemical sensors with three electrode systems (working, counter and reference electrodes) operate in an electrolyte coupled with an external potentiostat (see FIG. 3). Various designs have been reported for the electrochemical based sensors, but limited effort has been devoted to the development of array based sensors for the detection of multiple analytes. The problems with multi-sensor arrays are the cross-interference from other compounds, stability of the sensor element, etc. Biosensors have been reported using capture reagents. Devices and methods for detecting analytes using electrosensor having capture reagents, (World patent, WO0138873, 2001) but limited to the detection of a single analyte. Though different nanoscale materials like nanotubes, nanowires, nanoparticles have been used as substrates or binding agents, there has been a lack of a unified approach for the development of a matrix nanobiosensor, capable to multi-analyte detection. The electrochemical biosensing technique has great diversity in the detection of both liquid and gaseous analytes with accuracy in comparison with optical, dielectric, capacitive, resistance based techniques. The specific interaction of the analyte with the biological agent can be monitored with time electrochemically in the disclosed invention.

U.S. Pat. No. 6,656,712 describes the attachment of proteins to carbon nanotubes by incubation, without stirring. The biological macromolecule in solution is attached to the carbon nanotubes closed at their ends, under suitable temperature and pH conditions. The present invention uses an approach for the attachment of macromolecules, enzymes, proteins, antibodies, aptamers, nucleic acids, antigens, DNA, aptamers, ribozomes that includes electropolymerization with a conducting polymer matrix. An array of such sensors can be carried out within a few minutes and gives a stable framework for the electrochemical/biochemical reactions. The present invention also provides an efficient method of detection of both gaseous and liquid analytes by the use of a hydrophobic, semi-permeable membrane. The electrolyte can be wet (liquid phase) or dry (nafion, nanostructured silica, hydrogels and others) for the detection of chemical, biological warfare agents, gas detection, metabolic monitoring and other applications.

Linear Array Nanobiosensor:

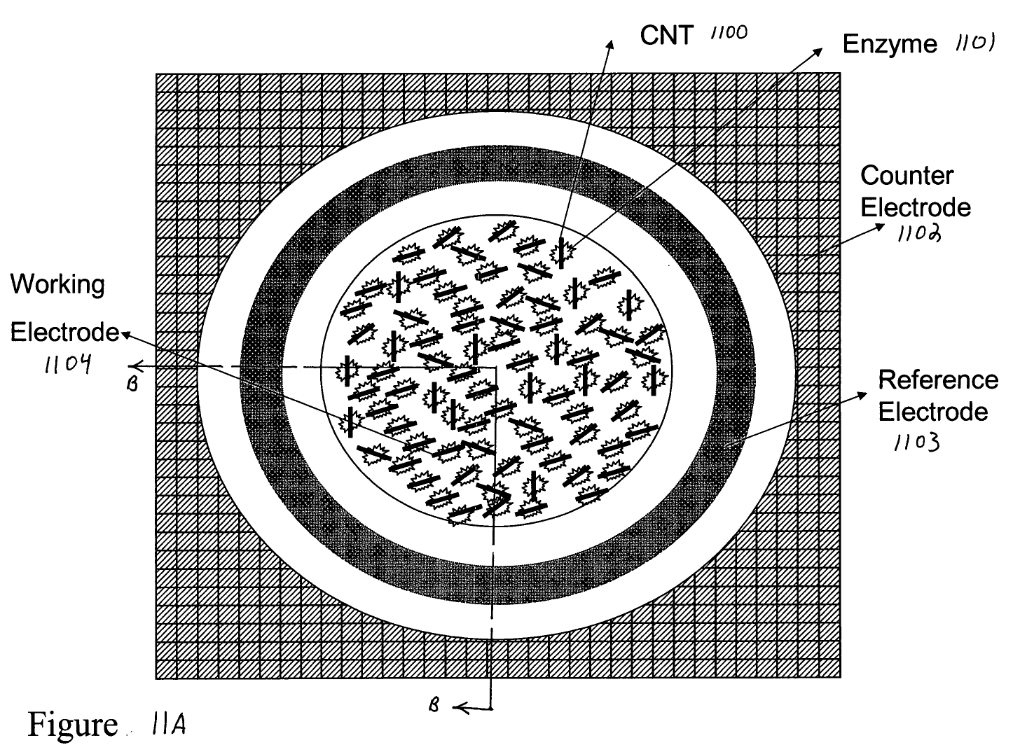

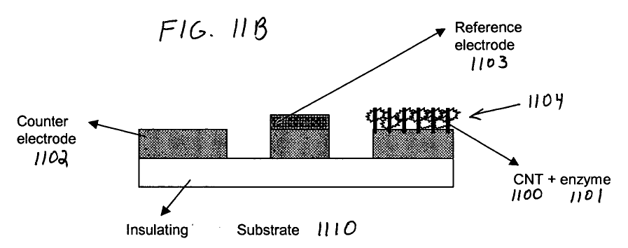

A design of a linear nanobiosensor in accordance with an embodiment of the present invention is shown in FIGS. 10, 11A and 11B. FIG. 11A is a top view, while FIG. 11B is a partial cross-sectional view of one of the electrode assemblies illustrated in FIG. 10. As described above, the sensor element is comprised of carbon nanotubes 1100, and conducting polymer and biological enzymes 1101. The sensing element is the working electrode 1104 supported on a silicon substrate 1110; other substrates like plastic (polymeric), glass, ceramic, ITO, kapton and printed circuit boards may also be used. An insulating layer used over the bare substrate can be metal nitride, metal oxide, polymer, etc. The reference electrode 1103 can be made up of Ag/AgCl (silver, silver chloride), SCE (standard calomel electrode), SHE (standard hydrogen electrode) or other standard reference electrodes. The counter electrode 1102 can be a conductive layer like gold, silver, copper, titanium, platinum, chromium, aluminum, etc. In the linear structure, insulating supports (not shown) may be provided to separate the reference 1103, working 1104 and counter 1102 electrodes, since it is necessary for the three electrodes to be separated from each other during the electrochemical process. The invention provides an efficient way for the detection of liquid and gaseous analytes. The gas sensor may use an enclosed semi-permeable, hydrophobic membrane (not shown), which can also be permi-selective to distinguish between different gases (electron donors and electron acceptors). The membrane can be incorporated on the counter/working electrode or can be separated from the system. The purpose of the membrane is to allow a one way entry for the gas into the working electrode for the electrochemical reaction. It is understood that the purpose of the reference electrode 1103 in the three electrode system is to maintain a stable potential around the working electrodes 1104.

FIGS. 10, 11A and 11B provide a design wherein the counter electrode 1102 is placed in the top of the linear system. It is generally desirable to place the counter electrode 1102 far from the working electrode 1104, with an area at least ten times that of the working electrode.

Active Matrix Array Nanobiosensor:

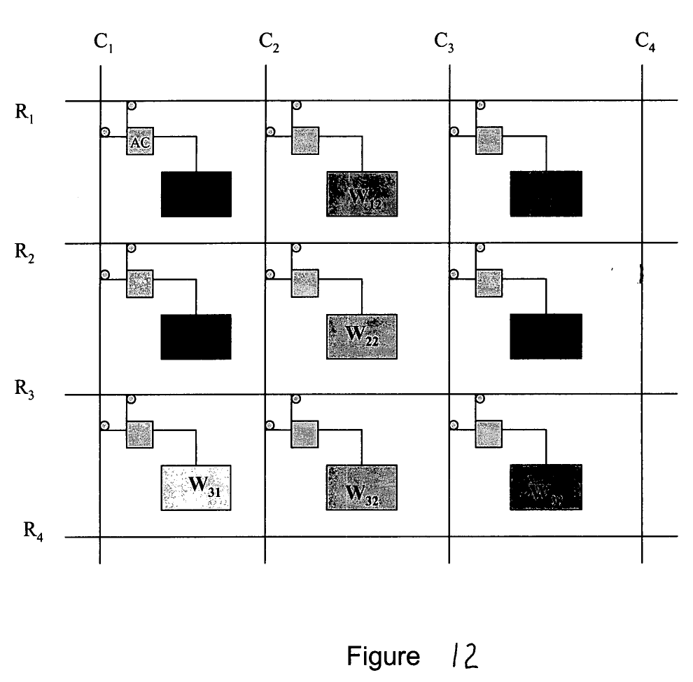

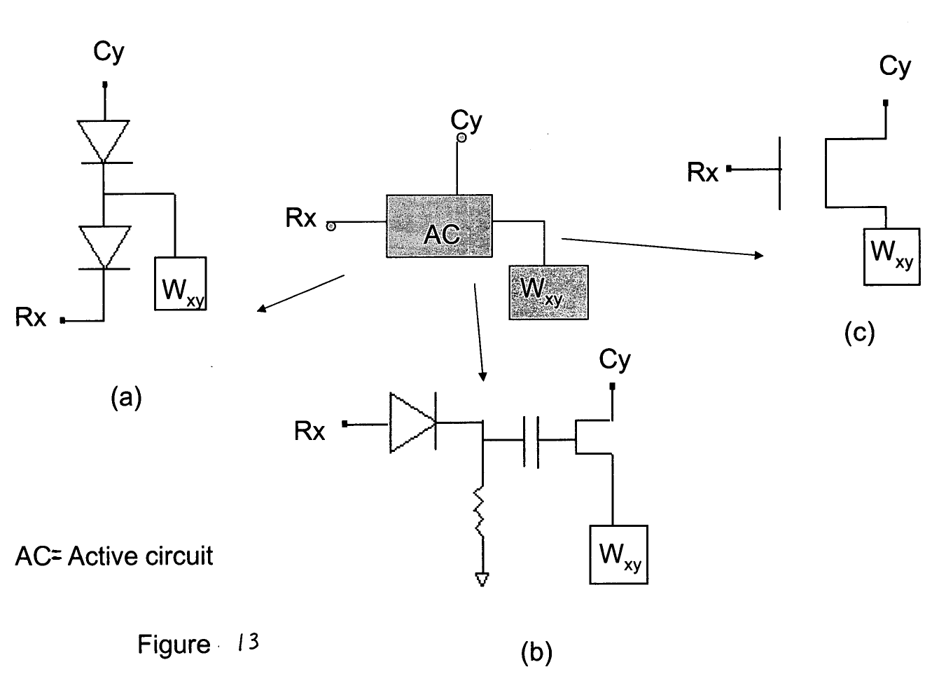

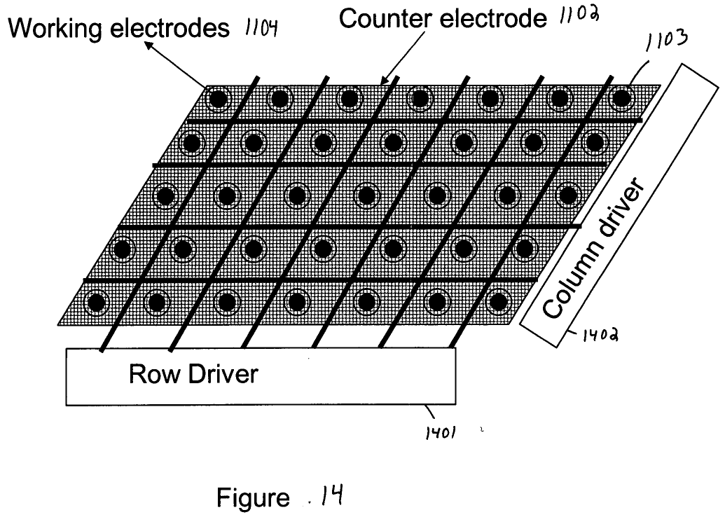

Active matrix circuits have been previously employed in liquid crystal displays (Azuma, Seiichiro, “Fabrication of Thin-Film Transistor for Active-Matrix Liquid-Crystal Display,” Patent: JP 2003100639, 2003; Hebiguchi, Hiroyuki, “Active Matrix Type LCD In Which a Pixel Electrodes Width Along a Scanning Line is Three Times Its Data Line Side Width,” U.S. Pat. No. 6,249,326, 2001). The effectiveness of the matrix nanobiosensor can be enhanced by employing a row-column addressable array which enables the development of an n×n matrix that can be fabricated in a cost effective and miniaturized fashion. FIG. 12 shows the top view of an exemplary 3×3 matrix array incorporating nine working electrodes (W₁₁ to W₃₃) (as described above with respect to FIG. 11), with each working electrode configuration driven by a row and a column driver (see FIG. 14). For example, the working electrode W₁₁ is driven by row 1 (R1) and column 1 (C₁). The sensor element (each working electrode configuration) also incorporates an active element (AC) for the row-column addressing, wherein R1 and C1 should be “turned on” to activate the working electrode W₁₁. The active matrix array design enables simultaneous driving of the multiple working electrodes, which cannot be achieved in the linear array design. For example, the voltage sweep of −1V to +1V at a scan rate of 50 mV/s in a cyclic voltammetric process takes 80 seconds for one electrode. The design shown in FIG. 2, wherein the working electrodes are arranged in a linear fashion, it would take 12 minutes to read the response of nine analytes. This presents a considerable disadvantage while sensing multiple analytes. FIG. 14 describes an active matrix configuration wherein the simultaneous driving of the working electrodes considerably shorten the time of detection. (For example, 80 seconds operating with cyclic voltammetry within the voltage sweep of −1V to +1V at a scan rate of 50 mV/s)”. This design provides considerable room for multiplexing the sensor elements and also for miniaturization of hundreds of sensor elements with microfabrication techniques. The active element (AC) is made up on an electrical circuit that can incorporate different configurations as shown in FIG. 13. The active circuit can constitute two diodes (FIG. 13a), a diode with a resistor connected to the capacitor (FIG. 13b), or a transistor (FIG. 13c). The invention is not limited to the above described active matrix components, but can be extended to any “active” electronic circuit that can address the row-column operation for the matrix array nanobiosensor. For any working electrode configuration (Wxy) shown in FIG. 13, the active matrix should be connected to a row (Rx) and column (Cy) as shown in FIG. 12.

The active working electrode components should incorporate the reference and the counter electrodes (see FIG. 11) for the electrochemical reactions. The electrochemical techniques include but are not limited to cyclic voltammetry, chronoamperometry, differential pulse voltammetry, linear sweep voltammetry, stripping voltammetry, AC voltammetry, AC impedance, etc. The sensor can be used to detect the analyte also by amperometric, potentiometric, conductometric, voltammetric methods or combinations thereof. FIG. 14 shows a cross-section view of an n×n array matrix nanobiosensor with the row 1401 and column 1402 drivers. The structure incorporates an n×n array of working electrodes 1104, each element incorporating the specific carbon nanotube 1100, conducting polymer, enzyme combination 1101 as described previously. This invention is not limited to the above mixture of components mentioned above but can be employed with carbon (all forms), noble metals (gold, platinum, etc.) and proteins, antibodies, nucleic acids (DNA, RNA), peptides, aptamers, aptazymes, proteins along with the different conducting polymer combinations.

Drive Electronics:

The drive electronics for the matrix array nanobiosensor is shown in FIG. 15. The active matrix array nanobiosensor incorporates a shift register 1401 that couples to the n×n array of working electrodes 1104 that is coupled to data in, clock and an enable. The working electrode arrays are coupled to a current to voltage (I/E) converter 1402, the reference electrodes to an electrometer circuit 1403 that is coupled to a control amplifier 1404 connected to the counter electrode 1102. The different components of the electronic circuit for the linear array sensor are similar to that described in FIG. 3. The reference electrodes 1103 are shown interdigitated with the working electrodes 1104, but different configurations are possible and are not limited to the invention. The voltage sweep typically used is around −1 V to +1 V with a scan rate of 50 mV with a step of 1 mV, though other voltage windows, scan rate and step sizes can be used for cyclic voltammetric measurements. The multi-channel output from the n×n arrays is coupled to a display device (not shown) or an alarm (not shown) to indicate the presence of the analyte. The design shown in FIG. 15 can be altered to drive n×n sensor elements simultaneously by building miniaturized circuitry (electrometer, I/E converter, control amplifier) for each of the sensor elements. The row and column drivers shown in FIG. 14 are addressable to any X-Y element in the sensor matrix. Each of the active elements 1405 (AC) can be controlled by a switch 1406 through the row-column addressing.

Although the present invention and its advantages have been described in detail, it should be understood that various changes, substitutions and alterations can be made herein without departing from the spirit and scope of the invention as defined by the appended claims.

1. A method of detecting multiple analytes using an array of nanobiosensors, wherein a plurality of nanobiosensors in the array have unique biological entities immobilized onto carbon nanotubes.

2. The method as recited in claim 1, wherein a first one of the plurality of nanobiosensors has a first biological entity immobilized onto carbon nanotubes, and wherein a second one of the plurality of nanobiosensors has a second biological entity immobilized onto carbon nanotubes, the first biological entity is unique relative to the second biological entity.

3. The method as recited in claim 2, wherein each nanobiosensor includes a working electrode having a biological entity immobilized onto carbon nanotubes, a reference electrode, and a counter electrode.

4. The method as recited in claim 3, wherein the electrodes are connected to circuitry for scanning each of the nanobiosensors.

5. An apparatus for detecting multiple analytes comprising an array of nanobiosensors, each comprising a biological entity immobilized onto carbon nanotubes.

6. The apparatus as recited in claim 5, wherein a plurality of the nanobiosensors in the array have unique biological entities.

7. The apparatus as recited in claim 6, wherein a first one of the plurality of nanobiosensors has a first biological entity immobilized onto carbon nanotubes, and wherein a second one of the plurality of nanobiosensors has a second biological entity immobilized onto carbon nanotubes, the first biological entity is unique relative to the second biological entity.

8. The apparatus as recited in claim 7, wherein each nanobiosensor includes a working electrode having a biological entity immobilized onto carbon nanotubes, a reference electrode, and a counter electrode.

9. The apparatus as recited in claim 8, wherein the electrodes are connected to circuitry for scanning each of the nanobiosensors.

10. The apparatus as recited in claim 8, wherein the reference electrode is in close proximity of the individual working electrodes and arranged in an active matrix configuration.

11. The apparatus as recited in claim 8, wherein the multiple working electrodes configured in an active matrix can be driven simultaneously to detect multiple analytes.
